(19) United States
(12) Patent Application Publication
(10) Pub. No.: US 2003/0171386 A1
(43) Pub. Date: Sep. 11, 2003

(54) Small molecules for the treatment of abnormal cell growth

(75) Inventors: Richard D. Connell, East Lyme, CT (US); John C. Kath, Waterford, CT (US); James D. Moyer, East Lyme, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/315,863

(22) Filed: Dec. 10, 2002

Related U.S. Application Data
(60) Provisional application No. 60/341,091, filed on Dec. 12, 2001.

(51) Int. Cl. A61K 31/517; C 07D 4 3/02

(52) U.S. Cl. 514/266.2; 514/266.21; 514/266.22; 544/284

(57) ABSTRACT

This invention relates to small molecules that are useful in the treatment of abnormal cell growth, such as cancer, in mammals. This invention also relates to a method of using such small molecules in the treatment of abnormal cell growth in mammals, especially humans, and to pharmaceutical compositions containing such compounds. The invention further relates to small molecules that are selective for erbB2 receptor over the erbB1 receptor, wherein said erbB2 inhibitor has a range of selectivities for erbB2 over erbB1 between 50-1500.

### CROSS-REFERENCE TO RELATED APPLICATION

This application claims the benefit of U.S. Provisional Patent Application No. 60/341,091 filed Dec. 12, 2001, the contents of which are hereby incorporated by reference in its entirety.

### BACKGROUND OF THE INVENTION

This invention relates to small molecules that are useful in the treatment of abnormal cell growth, such as cancer, in mammals. This invention also relates to a method of using such small molecules in the treatment of abnormal cell growth in mammals, especially humans, and to pharmaceutical compositions containing such compounds. The invention further relates to small molecules, which are potent and highly selective for the erbB2 tyrosine kinase receptor over its homologous family member, the erbB1 tyrosine kinase receptor.

It is known that a cell may become cancerous by virtue of the transformation of a portion of its DNA into an oncogene (i.e., a gene which, on activation, leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype.

Receptor tyrosine kinases are enzymes which span the cell membrane and possess an extracellular binding domain for growth factors such as epidermal growth factor, a transmembrane domain, and an intracellular portion which functions as a kinase to phosphorylate specific tyrosine residues in proteins and hence to influence cell proliferation. Receptor tyrosine kinases include c-erbB-2 (also known as erbB2 or HER2), c-met, tie-2, PDGFr, FGFr, VEGFR and EGFR (also known as erbB1 or HER1). It is known that such kinases are frequently aberrantly expressed in common human cancers such as breast cancer, gastrointestinal cancer such as colon, rectal or stomach cancer, leukemia, ovarian, bronchial or pancreatic cancer. More particularly, it has also been shown that epidermal growth factor receptor (EGFR), which possesses tyrosine kinase activity, is mutated and/or overexpressed in many human cancers such as brain, lung, squamous cell, bladder, gastric, breast, head and neck, oesophageal, gynecological and thyroid tumors.

Accordingly, it has been recognized that inhibitors of receptor tyrosine kinases are useful as selective inhibitors of the growth of mammalian cancer cells. For example, erbstatin, a tyrosine kinase inhibitor, selectively attenuates the growth in athymic nude mice of a transplanted human mammary carcinoma, which expresses epidermal growth factor receptor tyrosine kinase (EGFR) but is without effect on the growth of another carcinoma, which does not express the EGF receptor. Thus, the compounds of the present invention, which are selective inhibitors of certain receptor tyrosine kinases, are useful in the treatment of abnormal cell growth, in particular cancer, in mammals. European patent publications, namely EP 0 566 226 A1 (published Oct. 20, 1993), EP 0 602 851 A1(published Jun. 22, 1994), EP 0 635 507 A1 (published Jan. 25, 1995), EP 0 635 498 A1 (published Jan. 25, 1995), and EP 0 520 722 A1 (published Dec. 30, 1992), refer to certain bicyclic derivatives, in particular quinazoline derivatives, as possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 28, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are U.S. patent application Ser. Nos. 09/488,350 (filed Jan. 20, 2000) and 09/488,378 (filed Jan. 20, 2000), both of which are incorporated herein by reference in their entirety.

Particular tyrosine kinase receptors have been studied closely. For example, the EGFR family consists of four closely related receptors, identified as EGFR (erbB1), erbB2 (HER2), erbB3 (HER3) and erbB4 (HER4). It has also been found that the erbB2 receptor is overexpressed in human breast cancer and ovarian cancer (Slamon et al., Science, Vol. 244, pages 707-712, 1989). The erbB2 receptor is also highly expressed in a number of other cancers, such as prostate cancer (Lyne et al., Proceedings of the American Association for Cancer Research, Vol. 37, page 243, 1996) and gastric cancer (Yonemura et al., Cancer Research, Vol. 51, page 1034, 1991). Furthermore, studies have found that transgenic mice incorporating the erbB2 gene develop breast cancer (Guyre et al., Proceedings of the National Academy of Science, USA, Vol. 89, pages 10578-10582, 1992).

The following table shows the percentage of patients having HER2 overexpressed. Note that overexpression rates are variable depending the methodology and criteria used. The following literature references are incorporated in their entirety by reference into the present application: (i) S. Scholl, et al., Targeting HER2 in other tumor types, Annals of Oncology, 12 Suppl. 1, S81:S87, 2001; (ii) Koeppen H K, et al., Overexpression of HER2/neu in solid tumours: an immunohistochemical survey. Histopathology, February 2001; 38(2): 96-104; and (iii) Osman I, et al., Clinical Cancer Research, September 2001; 7(9):2643-7.

| | OVEREXPRESSION |
|---|---|
| CANCER | PERCENTAGE |
| Breast | 20-30% |
| Ovary | 18-43% |
| Non small cell lung | 13-55% |
| (NSCL) | |
| Colorectal (CRC) | 33-85% |
| Prostate | 5-46% |
| Bladder | 27-63% |
| Renal | 22-36% |
| Gastric | 21-64% |
| Endometrial | 10-52% |
| Head and Neck (H&N) | 16-50% |
| Esophageal | 10-26% |

One of the challenges encountered in the development of a small molecule selective erbB2 inhibitor is that the erbB2 receptor and its family member, the EGFR are highly homologous. Lack of specificity of inhibitors for the specific targeted family member has been found to lead to adverse events in clinical trials. In particular, in clinical trials conducted with compounds which are pan erbB inhibitors, i.e., compounds that inhibit all members of the EGFR family. For example, in clinical trials with pan erbB receptor inhibitors (CI-1033 and EKB-569) dermal toxicity in the form of a rash occurs. It is believed that the rash is due to the fact that the small molecules under study inhibit the erbB1 receptor tyrosine kinase leading to the adverse event. This theory has been supported by the fact that the same type of dermal toxicity was observed in clinical trials for compounds, which are selective erbB1 receptor inhibitors. For example, this adverse event was observed during clinical studies with the both Pfizer's small molecule erbB1 (EGFR) inhibitor CP-358,774 (now referred to as OSI-774 or Tarceva™) and AstraZeneca's small molecule EGFR inhibitor ZD1839 (Irressa™). Other compounds such as PKI-166, an erbB1 inhibitor from Novartis, has also been reported to produce a similar dermal toxicity in its Phase 1 clinical trial (2nd international anti-cancer Drug Discovery & Development summit: 2001, Princeton N.J.). Furthermore in studies with Imclone's tailor-made anti-erbB1 monoclonal antibody C-225 a similar rash was reported (2nd international anti-cancer Drug Discovery & Development summit: 2001, Princeton N.J.). Given the structural distinction between Tarceva, Iressa, PKI-166, and the monoclonal antibody it is now believed in the art that inhibitors of the erbB1 receptor tyrosine kinase may be the cause of the dermal toxicity seen in a significant percentage of the patients using these agents in the clinic. In contrast, in clinical trials of Genentech's (South San Francisco, Calif.) tailor-made monoclonal antibody HERCEPTIN™ for the erbB2 receptor tyrosine kinase no rash was observed. Accordingly, the ability of a small molecule to discriminate between the erbB2 and erbB1 receptor may minimize or eliminate the occurrence of adverse events observed in clinical trials. This would provide a dramatic improvement in the art. The disfiguring nature of the rash may lead to poor compliance in chemotherapy treatment.

While Herceptin provided a means of treating patients in need of erbB2-related therapies with an agent that avoids this erbB1-related dermal toxicity, there are significant drawbacks to this agent that limit its utility and general applicability. Herceptin carries a “Black Box” warning relating to cardiomyopathy and hypersensitivity reactions including anaphylaxis. These later events are related to the fact that Herceptin is an antibody.

Hence there is a compelling need for pharmaceutically relevant agents that can be used to treat erbB2-related disorders that avoid the erbB1-related dermal toxicity and the hypersensitivity reactions seen with monoclonal antibodies such as Herceptin. Furthermore, a selective erbB2 will be useful for the treatment of diseases in which the erbB2 receptor is overexpressed, such as breast carcinomas and ovarian cancer.

Gazit et al., in the Journal of Medicinal Chemistry, 1991, vol., 34, pages 1896-1907, refer to a number of tyrphostins, which were found to discriminate between the erbB1 receptor tyrosine kinase and erbB2 receptor tyrosine kinase. However, the vast majority of the compounds referred to in Gazit et al. were selective for the erbB1 receptor over the erbB2 receptor. Furthermore, the compounds identified by Gazit were not particularly potent for either the erbB1 or erbB2 receptor. More recently, WO 00/44728 (published Aug. 3, 2000) and WO 01/77107 (published Oct. 18, 2001) referred to compounds, which are useful as growth factor receptor tyrosine kinase (particularly HER2) inhibitors. It is highly desirable to have small molecule erbB2 inhibitors, which are able to selectively inhibit erbB2 over the other members of the erbB family, and in particular erbB1. The inventors of the present invention now provide small molecules, which are both potent and highly selective inhibitors of erbB2 receptor tyrosine kinase over the erbB1 receptor tyrosine kinase.

### SUMMARY OF THE INVENTION

The present invention relates to a small molecule erbB2 inhibitor, wherein said erbB2 inhibitor has a range of selectivities for erbB2 over erbB1 between 50-1500. In a preferred embodiment of the present invention the erbB2 inhibitor has a range of selectivities for erbB2 over erbB1 between 60-1200. In a more preferred embodiment of the present invention the erbB2 inhibitor has a range of selectivities for erbB2 over erbB1 between 80-1000. In an even more preferred embodiment of the present invention the erbB2 inhibitor has a range of selectivities for erbB2 over erbB1 between 90-500. In a most preferred embodiment of the present invention the erbB2 inhibitor has a range of selectivities for erbB2 over erbB1 between 100-300. In the most preferred embodiment of the present invention the erbB2 inhibitor has a range of selectivities for erbB2 over erbB1 between 110-200.

In another specific embodiment of the present invention the erbB2 inhibitor has an IC₅₀ of less than about 100 nM. In a more preferred embodiment of the present invention the erbB2 inhibitor has an IC₅₀ of less than about 50 nM.

In one preferred embodiment of the present invention the small molecule erbB2 inhibitor is selected from the group consisting of:

N-{3-[4-(5-Methyl-6-phenoxy-pyridin-3-ylamino)-quinazolin-6-yl]-prop-2-ynyl}-2-oxo-propionamide

E-cyclopropanecarboxylic acid (3-{4-[3-methyl-4-(pyridin-3-yloxy)-phenylamino]-quinazolin-6-yl}-allyl)-amide

2-methoxy-N-(3-{4-[4-(3-methoxy-phenoxy)-3-methyl-phenylamino]-quinazolin-6-yl}-prop-2-ynyl)-acetamide

E-cyclopropanecarboxylic acid (3-{4-[3-chloro-4-(6-methyl-pyridin-3-yloxy)-phenylamino]-quinazolin-6-yl}-allyl)-amide

E-N-(3-{4-[3-chloro-4-(6-methyl-pyridin-3-yloxy)-phenylamino]-quinazolin-6-yl}-allyl)-acetamide

E-5-methyl-isoxazole-3-carboxylic acid (3-{4-[3-methyl-4-(6-methyl-pyridin-3-yloxy)-phenylamino]-quinazolin-6-yl}-allyl)-amide

E-3-{4-[3-methyl-4-(pyridin-3-yloxy)-phenylamino]-quinazolin-6-yl}-allyl)-carbamic acid methyl ester

3-methoxy-pyrrolidine-1-carboxylic acid (1,1-dimethyl-3-{4-[3-methyl-4-(6-methyl-pyridin-3-yloxy)-phenylamino]-quinazolin-6-yl}-prop-2-ynyl)-amide

E-2-methoxy-N-(3-{4-[3-methyl-4-(6-methyl-pyridin-3-yloxy)-phenylamino]-quinazolin-6-yl}-allyl)-acetamide

1-ethyl-3-(3-{4-[3-methyl-4-(pyridin-3-yloxy)-phenylamino]-quinazolin-6-yl}-prop-2-ynyl)-urea

E-cyclopropanecarboxylic acid (3-{4-[3-methyl-4-(6-methyl-pyridin-3-yloxy)-phenylamino]-quinazolin-6-yl}-allyl)-amide

1-(3-{4-[3-chloro-4-(pyridin-3-yloxy)-phenylamino]-quinazolin-6-yl}-prop-2-ynyl)-3ethyl-urea

2-dimethylamino-N-(3-{4-[3-methyl-4-(pyridin-3-yloxy)-phenylamino]-quinazolin-6-yl}-prop-2-ynyl)-acetamide

3-methyl-4-(pyridin-3-yloxy)-phenyl]-(6-piperidin-4-ylethynyl-quinazolin-4-yl)-amine

(3-{4-[3-methyl-4-(pyridin-3-yloxy)-phenylamino]-quinazolin-6-yl}-prop-2-ynyl)-carbamic acid methyl ester

3-methyl-isoxazole-5-carboxylic acid (3-{4-[3-methyl-4-(6-methyl-pyridin-3-yloxy)-phenylamino]-quinazolin-6-yl}-prop-2-ynyl)-amide,

and the pharmaceutically acceptable salts, prodrugs and solvates of the foregoing compounds.

In a more preferred embodiment of the present invention the erbB2 inhibitor is selected from the group consisting of:

E-cyclopropanecarboxylic acid (3-{4-[3-methyl-4-(pyridin-3-yloxy)-phenylamino]-quinazolin-6-yl}-allyl)-amide

E-5-methyl-isoxazole-3-carboxylic acid (3-{4-[3-methyl-4-(6-methyl-pyridin-3-yloxy)-phenylamino]-quinazolin-6-yl}-allyl)-amide

E-(3-{4-[3-methyl-4-(pyridin-3-yloxy)-phenylamino]-quinazolin-6-yl}-allyl)-carbamic acid methyl ester

3-methoxy-pyrrolidine-1-carboxylic acid (1,1-dimethyl-3-{4-[3-methyl-4-(6-methyl-pyridin-3-yloxy)-phenylamino]-quinazolin-6-yl}-prop-2-ynyl)-amide

3-methyl-isoxazole-5-carboxylic acid (3-{4-[3-methyl-4-(6-methyl-pyridin-3-yloxy)-phenylamino]-quinazolin-6-yl}-prop-2-ynyl)-amide,

and the pharmaceutically acceptable salts, prodrugs and solvates of the foregoing compounds.

In a most preferred embodiment of the present invention the erbB2 inhibitor is selected from the group consisting of:

E-cyclopropanecarboxylic acid (3-{4-[3-methyl-4-(pyridin-3-yloxy)-phenylamino]-quinazolin-6-yl}-allyl)-amide

E-(3-{4-[3-methyl-4-(pyridin-3-yloxy)-phenylamino]-quinazolin-6-yl}-allyl)-carbamic acid methyl ester

and the pharmaceutically acceptable salts, prodrugs and solvates of the foregoing compounds.

The present invention also relates to a small molecule erbB2 inhibitor, wherein said erbB2 inhibitor has a range of selectivities for erbB2 over erbB1 between 50-1500 and inhibits growth of tumor cells which overexpress erbB2 receptor in a patient treated with a therapeutically effective amount of said erbB2 inhibitor.

In another embodiment of the present invention the erbB2 inhibitor has a range of selectivities for erbB2 over erbB1 between 60-1200 and inhibits growth of tumor cells which overexpress erbB2 receptor in a patient treated with a therapeutically effective amount of said erbB2 inhibitor.

In another embodiment of the present invention the erbB2 inhibitor has a range of selectivities for erbB2 over erbB1 between 80-1000 and inhibits growth of tumor cells which overexpress erbB2 receptor in a patient treated with a therapeutically effective amount of said erbB2 inhibitor.

In another embodiment of the present invention the erbB2 inhibitor has a range of selectivities for erbB2 over erbB1 between 90-500 and inhibits growth of tumor cells which overexpress erbB2 receptor in a patient treated with a therapeutically effective amount of said erbB2 inhibitor.

In a more preferred embodiment of the present invention the erbB2 inhibitor has a range of selectivities for erbB2 over erbB1 between 100-300 and inhibits growth of tumor cells which overexpresses erbB2 receptor in a patient treated with a therapeutically effective amount of said erbB2 inhibitor.

In a most preferred embodiment of the present invention the erbB2 inhibitor has a range of selectivities for erbB2 over erbB1 between 110-200 and inhibits growth of tumor cells which overexpresses erbB2 receptor in a patient treated with a therapeutically effective amount of said erbB2 inhibitor.

The present invention also relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 50-1500.

In another embodiment the present invention relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 60-1200.

In another embodiment the present invention relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 80-1000.

In another embodiment the present invention relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 90-500.

In yet another embodiment the present invention relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 100-300.

In a most preferred embodiment the present invention relates to a method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 110-200.

The present invention further relates to a method for the treatment of abnormal cell growth in a mammal comprising administering to said mammal an amount of an erbB2 inhibitor compound, which is selective for erbB2 over erbB1, that is effective in treating abnormal cell growth.

In one preferred embodiment of the present invention the abnormal cell growth is cancer.

In one embodiment of the present the cancer is selected is selected from lung cancer, non small cell lung (NSCL), bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), colorectal cancer (CRC), primary CNS lymphoma, spinal axis tumors, brain stem glioma, pituitary adenoma, or a combination of one or more of the foregoing cancers.

In a preferred embodiment of the present invention, cancer is selected from breast cancer, colon cancer, ovarian cancer, non small cell lung (NSCL) cancer, colorectal cancer (CRC), prostate cancer, bladder cancer, renal cancer, gastric cancer, endometrial cancer, head and neck cancer, and esophagel cancer.

In a more preferred embodiment of the present invention, the cancer is selected from renal cell carcinoma, gastric cancer, colon cancer, breast cancer, and ovarian cancer.

In a more preferred embodiment, the said cancer is selected from colon cancer, breast cancer or ovarian cancer.

Another embodiment of the present invention relates to method for the treatment of abnormal cell growth in a mammal which comprises administering to said mammal an amount of an erbB2 inhibitor, wherein said erbB2 inhibitor is selective for erbB2 over erbB1, that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

A preferred embodiment invention relates to a method for the treatment of abnormal cell growth in a mammal which comprises administering to said mammal an amount of an erbB2 inhibitor, wherein said erbB2 inhibitor is selective for erbB2 over erbB1, that is effective in treating abnormal cell growth in combination in combination with a cytotoxic.

In one preferred embodiment of the present invention the cytotoxic is Taxol® (paclitaxel).

The present invention further relates to a method for the treatment of abnormal cell growth in a mammal which comprises administering to said mammal an amount of a compound of claim 1 that is effective in treating abnormal cell growth in combination with a compound selected from the group consisting of Cyclophosphamide, 5-Fluorouracil, Floxuridine, Gemcitabine, Vinblastine, Vincristine, Daunorubicin, Doxorubicin, Epirubicin, Tamoxifen, Methylprednisolone, Cisplatin, Carboplatin, CPT-11, gemcitabine, paclitaxel, and docetaxel.

In one preferred embodiment, the invention relates to a method for the treatment of abnormal cell growth in a mammal which comprises administering to said mammal an amount of a compound of claim 1 that is effective in treating abnormal cell growth in combination with a compound selected from the group consisting Tamoxifen, Cisplatin, Carboplatin, paclitaxel and docetaxel.

The invention further relates to a pharmaceutical composition for the treatment of abnormal cell growth in a mammal comprising an amount of an erbB2 inhibitor, which is selective for erbB2 over erbB1, that is effective in treating abnormal cell growth, and a pharmaceutically acceptable carrier.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 50-1500 as measured by an in vitro cell assay.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 60-1200 as measured by an in vitro cell assay.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 80-1000 as measured by an in vitro cell assay.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 90-500 as measured by an in vitro cell assay.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 100-300 as measured by an in vitro cell assay.

The present invention also relates to a method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 110-200 as measured by an in vitro cell assay.

This invention also relates to a method for the treatment of abnormal cell growth in a mammal, including a human, comprising administering to said mammal an amount of an erbB2 inhibitor, as defined above, or a pharmaceutically acceptable salt, solvate or prodrug thereof, that is effective in treating abnormal cell growth. In one embodiment of this method, the abnormal cell growth is cancer, including, but not limited to, non small cell lung (NSCL) cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

This invention also relates to a method for the treatment of abnormal cell growth in a mammal which comprises administering to said mammal an amount of an erbB2 inhibitor, as defined above, or a pharmaceutically acceptable salt, solvate or prodrug thereof, that is effective in treating abnormal cell growth in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

This invention also relates to a pharmaceutical composition for the treatment of abnormal cell growth in a mammal, including a human, comprising an amount of an erbB2 inhibitor, as defined above, or a pharmaceutically acceptable salt, solvate or prodrug thereof, that is effective in treating abnormal cell growth, and a pharmaceutically acceptable carrier. In one embodiment of said composition, said abnormal cell growth is cancer, including, but not limited to, lung cancer, non small cell lung (NSCL), bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said pharmaceutical composition, said abnormal cell growth is a benign proliferative disease, including, but not limited to, psoriasis, benign prostatic hypertrophy or restinosis.

The invention also relates to a pharmaceutical composition for the treatment of abnormal cell growth in a mammal, including a human, which comprises an amount of an erbB2 inhibitor, as defined above, or a pharmaceutically acceptable salt, solvate or prodrug thereof, that is effective in treating abnormal cell growth in combination with a pharmaceutically acceptable carrier and an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for treating a mammal having cancer characterized by an overexpression of erbB2, comprising administering to the mammal a small molecule erbB2 inhibitor in an amount that is effective in treating said cancer characterized by the overexpression of erbB2, and said erbB2 inhibitor is selective for erbB2 over erbB1 at any of the ratios and with any of the IC₅₀ identified herein.

The invention also relates to a method for treating a mammal having a disease characterized by an overexpression of erbB2, comprising administering to the mammal a small molecule erbB2 inhibitor in an amount that is effective in treating a disease characterized by the overexpression of erbB2, and said erbB2 inhibitor is selective for erbB2 over erbB1 at any of the ratios and with any of the IC₅₀ identified herein.

The invention also relates to a method inducing cell death comprising exposing a cell which overexpresses erbB2 to an effective amount of an erbB1-sparing erbB2 inhibitor. In one embodiment the cell is a cancer cell in a mammal, preferably a human.

In another embodiment the present invention relates to a method inducing cell death comprising exposing a cell which overexpresses erbB2 to an effective amount of an erbB1-sparing erbB2 inhibitor and said method further comprises exposing the cell to a growth inhibitory agent.

In one preferred embodiment the cell is exposed to a chemotherapeutic agent or radiation.

The invention further relates to a method of treating cancer in a human, wherein the cancer expresses the erbB2 receptor, comprising administering to the human a therapeutically effective amount of an erbB2 inhibitor that has reduced affinity for the erbB1 receptor. In one preferred embodiment of the present invention the cancer is not characterized by overexpression of erbB1 receptor. In another preferred embodiment the cancer is characterized by overexpression of the erbB1 and erbB2 receptor.

This invention also relates to a method for the treatment of a disorder associated with angiogenesis in a mammal, including a human, comprising administering to said mammal an amount of an erbB2 inhibitor, as defined above, or a pharmaceutically acceptable salt, solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

This invention also relates to a method of (and to a pharmaceutical composition for) treating abnormal cell growth in a mammal which comprise an amount of an erbB2 inhibitor, as defined above, or a pharmaceutically acceptable salt, solvate or prodrug thereof, and an amount of one or more substances selected from anti-angiogenesis agents, signal transduction inhibitors, and antiproliferative agents, which amounts are together effective in treating said abnormal cell growth.

Anti-angiogenesis agents, such as MMP-2 (matrix-metalloprotienase 2) inhibitors, MMP-9 (matrix-metalloprotienase 9) inhibitors, and COX-II (cyclooxygenase II ) inhibitors, can be used in conjunction with an amount of an erbB2 inhibitor, as defined above, in the methods and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1.

More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Some specific examples of MMP inhibitors useful in combination with the compounds of the present invention are AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid;

3-exo-3-[4-(4-fluoro-phenoxy)-benzenesulfonylamino]-8-oxa-bicyclo[3.2.1]octane-3-carboxylic acid hydroxyamide;

(2R, 3R) 1-[4-(2-chloro-4-fluoro-benzyloxy)-benzenesulfonyl]-3-hydroxy-3-methyl-piperidine-2-carboxylic acid hydroxyamide;

4-[4-(4-fluoro-phenoxy)-benzenesulfonylamino]-tetrahydro-pyran-4-carboxylic acid hydroxyamide;

3-[[4-(4-fluoro-phenoxy)-benzenesulfonyl]-(1-hydroxycarbamoyl-cyclobutyl)-amino]-propionic acid;

4-[4-(4-chloro-phenoxy)-benzenesulfonylaminol-tetrahydro-pyran-4-carboxylic acid hydroxyamide;

3-[4-(4-chloro-phenoxy)-benzenesulfonylamino]-tetrahydro-pyran-3-carboxylic acid hydroxyamide;

(2R, 3R) 1-[4-(4-fluoro-2-methyl-benzyloxy)-benzenesulfonyl]-3-hydroxy-3-methyl-piperidine-2-carboxylic acid hydroxyamide;

3-[[4-(4-fluoro-phenoxy)-benzenesulfonyl]-(1-hydroxycarbamoyl-1-methyl-ethyl)-amino]-propionic acid;

3-[[4-(4-fluoro-phenoxy)-benzenesulfonyl]-(4-hydroxycarbamoyl-tetrahydro-pyran-4-yl)-amino]-propionic acid;

3-exo-3-[4-(4-chloro-phenoxy)-benzenesulfonylamino]-8-oxa-bicyclo[3.2.1]octane-3-carboxylic acid hydroxyamide;

3-endo-3-[4-(4-fluoro-phenoxy)-benzenesulfonylamino]-8-oxa-bicyclo[3.2.1]octane-3-carboxylic acid hydroxyamide; and

3-[4-(4-fluoro-phenoxy)-benzenesulfonylamino]-tetrahydro-furan-3-carboxylic acid hydroxyamide;

and pharmaceutically acceptable salts, solvates and prodrugs of said compounds.

The erbB2 compounds as defined above, and the pharmaceutically acceptable salts, solvates and prodrugs thereof, can also be used in combination with signal transduction inhibitors, such as VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

VEGF inhibitors, for example SU-5416 and SU-6668 (Sugen Inc. of South San Francisco, Calif., USA), can also be combined with a erbB2 compound as defined above. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used with the compounds of the present invention include inhibitors of the enzyme farnesyl protein transferase and inhibitors of the receptor tyrosine kinase PDGFr, including the compounds disclosed and claimed in the following U.S. patent applications Ser. Nos.: 09/221946 (filed Dec. 28, 1998); 09/454058 (filed Dec. 2, 1999); 09/501163 (filed Feb. 9, 2000); 09/539930 (filed Mar. 31, 2000); 09/202796 (filed May 22, 1997); 09/384339 (filed Aug. 26, 1999); and 09/383755 (filed Aug. 26, 1999); and the compounds disclosed and claimed in the following U.S. provisional patent applications: 60/168207 (filed Nov. 30, 1999); 60/170119 (filed Dec. 10, 1999); 60/177718 (filed Jan. 21, 2000); 60/168217 (filed Nov. 30, 1999), and 60/200834 (filed May 1, 2000). Each of the foregoing patent applications and provisional patent applications is herein incorporated by reference in their entirety.

An erbB2 inhibitor as define above may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors, for example the farnesyl protein transferase inhibitors described in the references cited in the “Background” section, supra. Specific CTLA4 antibodies that can be used in the present invention include those described in U.S. Provisional Application 60/113,647 (filed Dec. 23, 1998) which is herein incorporated by reference in its entirety.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; (4) any tumors that proliferate by receptor tyrosine kinases; (5) any tumors that proliferate by aberrant serine/threonine kinase activation; and (6) benign and malignant cells of other proliferative diseases in which aberrant serine/threonine kinase activation occurs..

A small molecule as used herein refers to non-DNA, non-RNA, non-polypeptide and non-monoclonal antibody molecules with a molecular weight of under 1000 AMV. Preferred small molecules are selective for erbB2 over erbB1 at a ratio of at least about 100:1.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “erbB1-sparing”, as used herein, unless otherwise indicated, means an inhibitor that demonstrates activity against various versions and homologs of the mamalian erbB2-related kinase, or cells expressing the erbB2 receptor with reduced or no activity against the corresponding erbB1-related kinases or cells. This reduction is expressed in the form of a selectivity ratio as defined previously.

The phrase “pharmaceutically acceptable salt(s)”, as used herein, unless otherwise indicated, includes salts of acidic or basic groups which may be present in the compounds of the present invention. The compounds of the present invention that are basic in nature are capable of forming a wide variety of salts with various inorganic and organic acids. The acids that may be used to prepare pharmaceutically acceptable acid addition salts of such basic compounds of are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)]salts. The compounds of the present invention that include a basic moiety, such as an amino group, may form pharmaceutically acceptable salts with various amino acids, in addition to the acids mentioned above.

Those compounds of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The compounds of the present invention have asymmetric centers and therefore exist in different enantiomeric and diastereomeric forms. This invention relates to the use of all optical isomers and stereoisomers of the compounds of the present invention, and mixtures thereof, and to all pharmaceutical compositions and methods of treatment that may employ or contain them. The compounds of the present invention may also exist as tautomers. This invention relates to the use of all such tautomers and mixtures thereof.

The subject invention also includes isotopically-labelled compounds, and the pharmaceutically acceptable salts, solvates and prodrugs thereof, which are identical to those recited above, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of identified above and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating bacterial infections through administering prodrugs of compounds of the present invention. Compounds of present invention may have free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the present invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

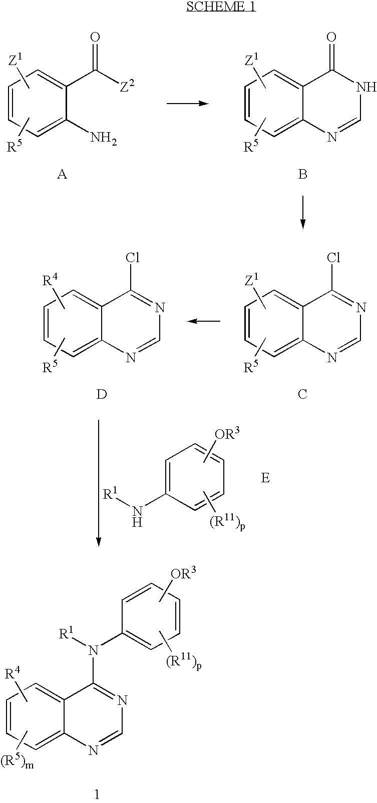

### DETAILED DESCRIPTION OF THE INVENTION

General synthetic methods which may be referred to for preparing the compounds of the present invention are provided in U.S. Pat. No. 5,747,498 (issued May 5, 1998), U.S. patent application Ser. No. 08/953078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in U.S. patent application Ser. Nos. 09/488,350 (filed Jan. 20, 2000) and 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem. 1986, 51, 5, p. 616. Palladium-catalyzed boronic acid couplings are described in Miyaura, N., Yanagi, T., Suzuki, A. Syn. Comm. 1981, 11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

Alternatively terminal alkynes may be prepared by a two step procedure. First, the addition of the lithium anion of TMS (trimethylsilyl) acetylene to an appropriately substituted/protected aldehyde as in: Nakatani, K. et. al. Tetrahedron, 49, 9, 1993, 1901. Subsequent deprotection by base may then be used to isolate the intermediate terminal alkyne as in Malacria, M.; Tetrahedron, 33, 1977, 2813; or White, J. D. et. al. Tet. Lett., 31, 1, 1990, 59.

Starting materials, the synthesis of which is not specifically described above, are either commercially available or can be prepared using methods well known to those of skill in the art.

In each of the reactions discussed or illustrated in the Schemes above, pressure is not critical unless otherwise indicated. Pressures from about 0.5 atmospheres to about 5 atmospheres are generally acceptable, and ambient pressure, i.e., about 1 atmosphere, is preferred as a matter of convenience.

With reference to Scheme 1 above, the compound of formula 1 may be prepared by coupling the compound of formula D wherein R⁴ and R⁵ are defined above, with an amine of formula E wherein R¹, R³ and R¹¹ are as defined above, in an anhydrous solvent, in particular a solvent selected from DMF (N,N-dimethylformamide), DME (ethylene glycol dimethyl ether), DCE (dichloroethane) and t-butanol, and phenol, or a mixture of the foregoing solvents, a temperature within the range of about 50-150° C. for a period ranging from 1 hour to 48 hours. The heteroaryloxyanilines of formula E may be prepared by methods known to those skilled in the art, such as, reduction of the corresponding nitro intermediates. Reduction of aromatic nitro groups may be performed by methods outlined in Brown, R. K., Nelson, N. A. J. Org. Chem. 1954, p. 5149; Yuste, R., Saldana, M, Walls, F., Tet. Lett. 1982, 23, 2, p. 147; or in WO 96/09294, referred to above. Appropriate heteroaryloxy nitrobenzene derivatives may be prepared from halo nitrobenzene precursors by nucleophilic displacement of the halide with an appropriate alcohol as described in Dinsmore, C. J. et. al., Bioorg. Med. Chem. Lett., 7, 10, 1997, 1345; Loupy, A. et. al., Synth. Commun., 20, 18, 1990, 2855; or Brunelle, D. J., Tet. Lett., 25, 32, 1984, 3383. Compounds of formula E in which R¹ is a C₁-C₆ alkyl group may be prepared by reductive animation of the parent aniline with R¹CH(O). The compound of formula D may be prepared by treating a compound of formula C, wherein Z¹ is an activating group, such as bromo, iodo, —N₂, or —OTf (which is —OSO₂CF₃), or the precursor of an activating group such as NO₂, NH₂ or OH, with a coupling partner, such as a terminal alkyne, terminal alkene, vinyl halide, vinyl stannane, vinylborane, alkyl borane, or an alkyl or alkenyl zinc reagent. The compound of formula C can be prepared by treating a compound of formula B with a chlorinating reagent such as POCl₃, SOCl₂ or ClC(O)C(O)Cl/DMF in a halogenated solvent at a temperature ranging from about 60° C. to 150° C. for a period ranging from about 2 to 24 hours. Compounds of formula B may be prepared from a compound of formula A wherein Z¹ is as described above and Z² is NH₂, C₁-C₆ alkoxy or OH, according to one or more procedures described in WO 95/19774, referred to above.

Any compound described above can be converted into another compound by standard manipulations to the R⁴ group. These methods are known to those skilled in the art and include a) removal of a protecting group by methods outlined in T. W. Greene and P. G. M. Wuts, “Protective Groups in Organic Synthesis”, Second Edition, John Wiley and Sons, New York, 1991; b) displacement of a leaving group (halide, mesylate, tosylate, etc) with a primary or secondary amine, thiol or alcohol to form a secondary or tertiary amine, thioether or ether, respectively; c) treatment of phenyl (or substituted phenyl) carbamates with primary of secondary amines to form the corresponding ureas as in Thavonekham, B et. al. Synthesis (1997), 10, p1189; d) reduction of propargyl or homopropargyl alcohols or N-BOC protected primary amines to the corresponding E-allylic or E-homoallylic derivatives by treatment with sodium bis(2-methoxyethoxy)aluminum hydride (Red-Al) as in Denmark, S. E.; Jones, T. K. J. Org. Chem. (1982) 47, 4595-4597 or van Benthem, R. A. T. M.; Michels, J. J.; Speckamp, W. N. Synlett (1994), 368-370; e) reduction of alkynes to the corresponding Z-alkene derivatives by treatment hydrogen gas and a Pd catalyst as in Tomassy, B. et. al. Synth. Commun. (1998), 28, p1201 f) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; g) reductive amination of a primary or secondary amine using R¹CH(O); and h) treatment of alcohols with an isocyanate, acid chloride (or other activated carboxylic acid derivative), alkyl/aryl chloroformate or sulfonyl chloride to provide the corresponding carbamate, ester, carbonate or sulfonic acid ester.

The compounds of the present invention may have asymmetric carbon atoms. Diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of present invention that are basic in nature are capable of forming a wide variety of different salts with various inorganic and organic acids. Although such salts must be pharmaceutically acceptable for administration to animals, it is often desirable in practice to initially isolate the compound of present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product. Since a single compound of the present invention may include more than one acidic or basic moieties, the compounds of the present invention may include mono, di or tri-salts in a single compound.

The compounds of the present invention are potent inhibitors of the erbB family of oncogenic and protooncogenic protein tyrosine kinases, in particular erbB2, and thus are all adapted to therapeutic use as antiproliferative agents (eq., anticancer) in mammals, particularly in humans. In particular, the compounds of the present invention are useful in the prevention and treatment of a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.q., psoriasis) and benign hyperplasia of the prostate (e.g., BPH). It is, in addition, expected that a compound of the present invention may possess activity against a range of leukemias and lymphoid malignancies.

The compounds of the present invention may also be useful in the treatment of additional disorders in which aberrant expression ligand/receptor interactions or activation or signalling events related to various protein tyrosine kinases, are involved. Such disorders may include those of neuronal, glial, astrocytal, hypothalamic, and other glandular, macrophagal, epithelial, stromal, and blastocoelic nature in which aberrant function, expression, activation or signalling of the erbB tyrosine kinases are involved. In addition, the compounds of the present invention may have therapeutic utility in inflammatory, angiogenic and immunologic disorders involving both identified and as yet unidentified tyrosine kinases that are inhibited by the compounds of the present invention.

The ability of small molecules, their pharmaceutically acceptable salts, prodrugs and solvates to inhibit the erbB2 tyrosine kinase receptor and the erbB1 tyrosine kinase receptor, and consequently, demonstrate their effectiveness for treating diseases characterized by erbB2 is shown by the following in vitro cell assay test.

The in vitro activity of small molecule compounds as erbB kinase inhibitors in intact cells may be determined by the following procedure. Cells, for example 3T3 cells transfected with human EGFR (Cohen et al. J. Virology 67:5303, 1993) or with chimeric EGFR/erbB2 kinase (EGFR extracellular/erbB2 intracellular, Fazioli et al. Mol. Cell. Biol. 11: 2040, 1991) are plated in 96-well plates at 12,000 cells per well in 100 μl medium (Dulbecco's Minimum Essential Medium (DMEM) with 5% fetal calf serum, 1% pen/streptomycin, 1% L-glutamine) and incubated at 37° C., 5% CO₂ Test compounds are solubilized in DMSO at a concentration of 10 mM, and tested at final concentrations of 0, 0.3 μM, 1 μM, 0.3 μM, 0.1 μM and 10 μM in the medium. The cells are incubated at 37° C. for 2 h. EGF (40 ng/ml final) is added to each well and cells incubate at room temperature for 15 min followed by aspiration of medium, then 100 μl/well cold fixative (50% ethanol/50% acetone containing 200 micromolar sodium orthovanadate) is added. The plate is incubated for 30 min at room temperature followed by washing with wash buffer (0.5% Tween 20 in phosphate buffered saline). Blocking buffer (3% bovine serum albumin, 0.05% Tween 20, 200 μM sodium orthovanadate in phosphate buffered saline, 100 μl/well) is added followed by incubation for 2 hours at room temperature followed by two washes with wash buffer. PY54 monoclonal anti-phosphotyrosine antibody directly conjugated to horseradish peroxidase (50 μl/well, 1 μg/ml in blocking buffer) or blocked conjugate (1 μg/ml with 1 mM phosphotyrosine in blocking buffer, to check specificity) is added and the plates incubated for 2 hours at room temperature. The plate wells are then washed 4 times with wash buffer. The colorimetric signal is developed by addition of TMB Microwell Peroxidase Substrate (Kirkegaard and Perry, Gaithersburg, Md.), 50 μl per well, and stopped by the addition of 0.09 M sulfuric acid, 50 μl per well. Absorbance at 450 nM represents phosphotyrosine content of proteins. The increase in signal in EGF-treated cells over control (non-EGF treated) represents the activity of the EGFR or EGFR/chimera respectively. The potency of an inhibitor is determined by measurement of the concentration of compound needed to inhibit the increase in phosphotyrosine by 50% (IC₅₀) in each cell line. The selectivity of the compounds for erbB2 vs. EGFR is determined by comparison of the IC₅₀ for the EGFR transfectant vs. that for the erbB2/EGFR chimera transfectant. Thus, for example, a compound with an IC₅₀ of 100 nM for the EGFR transfectant and 10 nM for the erbB2/EGFR chimera transfectant is considered 10-fold selective for erbB2 kinase.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

Method A: Synthesis of [3-Methyl-4-(pyridin-3-yloxy)-phenyl]-(6-piperidin-4-ylethynyl-quinazolin-4-yl)-amine (1):

4-(4-Chloro-quinazolin-6-ylethynyl)-piperidine-1-carboxylic acid tert-butyl ester: A mixture of 4-ethynyl-piperidine-1-carboxylic acid tert-butyl ester (1.12 g, 5.35 mmol), 4-chloro-6-iodoquinazoline (1.35 g, 4.65 mmol), dichlorobis(triphenylphosphine) palladium(II) (0.16 g, 0.23 mmol), copper(I) iodide (0.044 g, 0.23 mmol), and diisopropylamine (0.47 g, 4.65 mmol) in anhydrous THF (20 mL) was stirred at room temperature under nitrogen for 2 hours. After concentration, the residue was dissolved in CH₂Cl₂ (100 mL), washed with aqueous NH₄Cl and brine, dried over sodium sulfate, and concentrated to give the crude product as brown oil. Purification by silica gel column using 20% EtOAc in hexane afforded 1.63 g (94%) of the title compound as a sticky, yellow oil: ¹H NMR (CDCl₃) δ1.45 (s, 9H), 1.67-1.75 (m, 2H), 1.87-1.92 (m, 2H), 2.84 (m, 1H), 3.20-3.26 (m, 2H), 3.78 (br d, 2H), 7.88 (dd, 1H), 7.97 (d, 1H), 8.26 (d, 1H), 9.00 (s, 1H).

[3-Methyl-4-(pyridin-3-yloxy)-phenyl]-(6-piperidin-4-ylethynyl-quinazolin-4-yl)-amine: 4-(4-Chloro-quinazolin-6-ylethynyl)-piperidine-1-carboxylic acid tert-butyl ester (80 mg, 0.21 mmol) and 3-Methyl-4-(pyridin-3-yloxy)-phenylamine (43 mg, 0.21 mmol) were mixed together in tert-butanol (1 mL) and dichloroethane (1 mL) and heated in a sealed vial at 90° C. for 20 minutes. The reaction was cooled down and HCl (gas) was bubbled through for 5 minutes. EtOAC was then added whereupon yellow precipitation occurred. The precipitate was collected and dried to afford the desired product [3-Methyl-4-(pyridin-3-yloxy)-phenyl]-(6-piperidin-4-ylethynyl-quinazolin-4-yl)-amine as a yellow solid (96 mg, 95%). ¹H NMR (CDCl₃) δ2.01 ((m, 2H), 2.22 (m, 2H), 2.35(s, 3H), 3.20 (m, 2H), 3.45(m, 2H), 7.28 (d, 1H, J=8.7 Hz), 7.75(dd, 3H, J1=8.7, J2=8.7 Hz), 8.06 (dd, J=8.7), 8.10 (dd, J1=J2=8.7 Hz), 8.17 (m, 1 H), 8.60 (d, 1H, J=5.4 Hz), 8.80 (s, 1H), 8.89 (s, 1H). MS: M+1, 436.6.

Method B: Synthesis of 2-Chloro-N-(3-{4-[3-methyl-4-(pyridin-3-yloxy)-phenylamino]-quinazolin-6-yl}-prop-2-ynyl)-acetamide (2):

2-Chloro-N-[3-(4-chloro-quinazolin-6-yl)-prop-2-ynyl]-acetamide: 2-Chloro-N-prop-2-ynyl-acetamide (385 mg; 2.93 mmol) and 4-chloro-6-iodoquinazoline (850 mg; 1 equiv.) were dissolved in dry THF and diisopropylamine (296 mg; 0.41 mL; 1 equiv.). To this mixture was added 0.04 equivalents of copper iodide (22 mg) and Pd(PPh₃)₂Cl₂ (82 mg). The reaction was stirred at room temperature under a nitrogen atmosphere overnight (−20 hrs). The solvent was then removed in vacuo and the residue dissolved in CH₂Cl₂. This solution was transferred to a separatory funnel and washed with 1× saturated NH₄Cl, brine, dried over Na₂SO₄ and the solvent removed in vacuo. The product was purified by silica gel chromatography eluting with 1:1 Hexanes/EtOAc and collecting fractions with an Rf=0.25. 2-Chloro-N-[3-(4-chloro-quinazolin-6-yl)-prop-2-ynyl]-acetamide was obtained as an off white solid (454 mg; 53%). ¹H NMR (400 MHz; CDCl₃) δ4.12 (2H, s), 4.40 (2H, d, J=5.2 Hz), 7.91-7.93 (1H, dd, J=2, 6.8 Hz), 8.00 (1H, d, J=8.4 Hz), 8.34 (1H, d, J=1.6 Hz), 9.03 (1H, s). Irms (M+): 294.0, 296.0, 298.1.

2-Chloro-N-(3-{4-[3-methyl-4-(pyridin-3-yloxy)-phenylamino]-quinazolin-6-yl}-prop-2-ynyl)-acetamide: A mixture of 2-Chloro-N-[3-(4-chloro-quinazolin-6-yl)-prop-2-ynyl]-acetamide (0.90 g, 3.05 mmol) and 3-Methyl-4-(pyridin-3-yloxy)-phenylamine (0.61 g, 3.05 mmol) in ^{t}BuOH/DCE (5.0/5.0 mL) was refluxed under nitrogen for 40 minutes and concentrated. The residue was dissolved in MeOH (2.0 mL) and added to EtOAc with vigorous stirring to precipitate the HCl salt product as tan solid which was collected by vacuum-filtration, rinsed with EtOAc, and further dried to give 1.24 g (82%) of 2-Chloro-N-(3-{4-[3-methyl-4-(pyridin-3-yloxy)-phenylamino]-quinazolin-6-yl}-prop-2-ynyl)-acetamide: ¹H NMR (CD₃OD) δ2.27 (s, 3H), 4.09 (s, 2H), 4.29 (s, 2H), 7.07 (d, 1H), 7.51 (m, 2H), 7.60 (d, 1H), 7.70 (s, 1H), 7.78 (d, 1H), 8.05 (d, 1H), 8.32 (m, 2H), 8.67 (s, 1H), 8.75 (s, 1H); MS m/z (MH⁺) 458.0.

Method C: Synthesis of 2-Dimethylamino-N-(3-{4-[3-methyl-4-(pyridin-3-yloxy)-phenylamino]-quinazolin-6-yl}-prop-2-ynyl)-acetamide (3):

2-Dimethylamino-N-(3-{4-[3-methyl-4-(pyridin-3-yloxy)-phenylamino]-quinazolin-6-yl}-prop-2-ynyl)-acetamide: To a solution of 2-Chloro-N-(3-{4-[3-methyl-4-(pyridin-3-yloxy)-phenylamino]-quinazolin-6-yl}-prop-2-ynyl)-acetamide (99 mg, 0.20 mmol) in MeOH (5 mL) was added a solution dimethylamine in THF (2 mL, 4.0 mmol). The resulting solution was refluxed under nitrogen for 1 hour. After concentration, the residue was further dried, dissolved in MeOH (1.0 mL), and treated with HCl gas for 3 minutes. The resulting solution was added to EtOAc with vigorous stirring to precipitate the HCl salt product as yellow solid which was collected by vacuum-filtration, rinsed with EtOAc, and further dried to give 110 mg (99%) of the title compound. ¹H NMR (CD₃OD) δ2.30 (s, 3H), 2.96 (s, 6H), 4.03 (s, 2H), 4.37 (s, 2H), 7.27 (d, 1H), 7.72 (dt, 1H), 7.81(m, 1H), 7.84 (d, 1H), 8.03 (dd, 1H), 8.06 (d, 1H), 8.13 (dd, 1H), 8.59 (d, 1H), 8.68 (s, 1H), 8.81 (s, 1H), 8.84 (s, 1H); MS m/z (MH⁺) 467.3.

Method D: Synthesis of 1-(3-{4-[3-Chloro-4-(6-methyl-pyridin-3-yloxy)-phenylamino]-quinazolin-6-yl}-prop-2-ynyl)-3-methyl-urea (4):

1-{4-[3-Chloro-4-(6-methyl-pyridin-3-yloxy)-phenylamino]-quinazolin-6-yl}-prop-2-ynyl)-3-methyl-urea: A mixture of (3-{4-[3-Chloro-4-(6-methyl-pyridin-3-yloxy)-phenylamino]-quinazolin-6-yl}-prop-2-ynyl)-carbamic acid phenyl ester (0.1 g, 0.18 mmol) prepared by Method B, methyl amine (2.0M methanol solution, 1 mL, 2 mmol) and DMSO (0.5 mL) was stirred at 80° C. overnight. The solvents were removed under vacuum (GeneVac HT-8) and the residue was re-dissolved in MeOH (˜1 mL). HCI gas was bubbled through the solution and EtOAc resulting in precipitation of the desired product. The title compound (80 mg, 90% yield) was obtained by filtration as a yellow solid. ¹H NMR (400 MHz, CD₃OD) δ2.72 (3H, s), 2.76 (3H, s), 4.19 (2H, s), 7.49 (1H, d, J=9 Hz), 7.84 (1H, d, J=2 Hz), 7.86 (1H, d, J=2 Hz), 7.92 (1H, d, J=9 Hz), 8.12 (2H, m, J=2 Hz), 8.16 (1H, d, J=2.4 Hz), 8.60 (1H, d, J=3.2 Hz), 8.74 (1H, d, J=1.2 Hz), 8.87 (1H, s ). LRMS (M⁺): 473.0, 475.0, 476.0.

Method E: Synthesis of 3-{4-[3-Methyl-4-(pyridin-3-yloxy)-phenylamino]-quinazolin-6-yl}-prop-2-en-1-ol (5):

3-{4-[3-Methyl-4-(pyridin-3-yloxy)-phenylamino]-quinazolin-6-yl}-prop-2-en-1-ol. To a solution of 0.56 g (1.47 mmol) of 3-{4-[3-methyl-4-(pyridin-3-yloxy)-phenylamino]-quinazolin-6-yl}-prop-2-yn-1-ol (prepared by Method B) in 6 mL of dry tetrahydrofuran at 0° C. was added 0.73 mL of a 65% weight toluene solution of sodium bis(2-methoxyethoxy)aluminum hydride (Red-Al, 2.35 mmol) in 1 mL of THF. The reaction was stirred at room temperature for 3 hours. Upon recooling to 0° C. an additional 0.73 mL of the Red-Al solution in 1 mL of THF was added. After stirring for 1 hour at room temperature, the mixture was quenched with the dropwise addition of 10% aqueous potassium carbonate and extracted with ethyl acetate. The organic extracts were dried over sodium sulfate, filtered and evaporated to give 650 mg. Chromatography on 90 g silica gel, eluting with 96:4:0.1 chloroform/methanol/concentrated ammonium hydroxide afforded 268 mg of the title compound. ¹H NMR (d₆ DMSO): δ9.79 (s, 1), 8.57 (m, 2), 8.35 (m, 2), 8.01 (m, 1), 7.80 (m, 3), 7.41 (m, 1), 7.29 (m, 1), 7.07 (d, J=8.7 Hz, 1), 6.77 (d, J=16.2 Hz, 1), 6.67 (m, 1), 5.04 (t, J=5.6 Hz, 1), 4.23 (m, 2), 2.23 (s, 3).

Method F: Synthesis of [3-Methyl-4-(pyridin-3-yloxy)-phenyl]-[6-(3-morpholin-4-yl-propenyl)-quinazolin-4-yl]-amine (6):

[3-Methyl-4-(pyridin-3-yloxy)-phenyl]-[6-(3-morpholin-4-yl-propenyl)-quinazolin-4-yl]-amine. To a suspension of 0.035 g (0.091 mmol) of 3-{4-[3-methyl-4-(pyridin-3-yloxy)-phenylamino]-quinazolin-6-yl}-prop-2-en-1-ol in 0.5 mL of methylene chloride and 1 mL of ethylene dichloride was added 1 mL of thionyl chloride. The reaction was heated at 100° C. for 1 hour and the solvents were evaporated to provide [6-(3-chloro-propenyl)-quinazolin-4-yl]-[3-methyl-4-(pyridin-3-yloxy)-phenyl]-amine [MS: M⁺403.1] which was dissolved in THF and used directly in the next reaction. To the solution of [6-(3-chloro-propenyl)-quinazolin-4-yl]-[3-methyl-4-(pyridin-3-yloxy)-phenyl]-amine was added 0.10 mL of morpholine and 0.044 mL of triethylamine. The mixture was heated at 85° C. for 16 hours, cooled to room temperature, and partitioned between 10% aqueous potassium carbonate and ethyl acetate. The aqueous layer was further extracted with ethyl acetate and the combined organics were dried and evaporated to yield 57 mg of material. The product was purified on a silica gel prep plate, eluting with 96:4:0.1 chloroform/methanol/concentrated ammonium hydroxide to afford 26 mg of the title compound; ¹H NMR (CDCl₃): δ8.71 (s, 1), 8.33 (m, 2), 7.94 (s, 1), 7.80 (m, 2), 7.69 (s, 1), 7.58 (m, 1), 7.20 (m, 1), 6.94 (d, J=8.7 Hz, 1), 6.68 (d, J=15.8 Hz, 1) 6.46 (m, 1), 3.79 (m, 4), 3.26(m, 2), 2.63 (m, 4), 2.25 (s, 3).

Method G: Synthesis of E-N-(3-{4-[3-Chloro-4-(6-methyl-pyridin-3-yloxy)-phenylamino]-quinazolin-6-yl}-allyl)-acetamide (7):

E-(3-{4-[3-chloro-4-(6-methyl-pyridin-3-yloxy)-phenylamino]-quinazolin-6-yl}-allyl)-carbamic acid tert-butyl ester: To a solution of 7.53 mL of a 65% weight toluene solution of sodium bis(2-methoxyethoxy)aluminum hydride (Red-Al, 24.2 mmol) in 90 mL of tetrahydrofuran at 0° C. was added 5.0 g of (3-{4-[3-chloro-4-(6-methyl-pyridin-3-yloxy)-phenylamino]-quinazolin-6-yl}-prop-2-ynyl)-carbamic acid tert-butyl ester as a solid. The reaction was stirred at 0° C. for 2 hours, quenched with 10% aqueous potassium carbonate and extracted with ethyl acetate. The combined organics were dried and evaporated. The crude material was purified on 115 g of silica gel, eluting with 80% ethyl acetate/hexanes to afford 4.42 g of E-(3-{4-[3-chloro-4-(6-methyl-pyridin-3-yloxy)-phenylamino]-quinazolin-6-yl}-allyl)-carbamic acid tert-butyl ester. ¹H NMR (CDCl₃): δ8.66 (s, 1), 8.24 (m, 1), 8.03 (m, 2), 7.77−7.65 (m, 3), 7.13 (m, 2), 6.97 (d, J=8.7 Hz, 1), 6.54 (d, 1), 6.35 (m, 1), 4.9 (m, 1), 3.90 (m, 2), 2.52 (s, 3), 1.46 (s, 9).

E-[6-(3-amino-propenyl)-quinazolin-4-yl]-[3-chloro-4-(6-methyl-pyridin-3-yloxy)-phenyl]-amine. To a solution of 4.42 g of E-(3-{4-[3-chloro-4-(6-methyl-pyridin-3-yloxy)-phenylamino]-quinazolin-6-yl}-allyl)-carbamic acid tert-butyl ester in 21 mL of tetrahydrofuran was added 21 mL of 2 N hydrochloric acid. The mixture was heated at 60° C. for 3 hours, cooled to room temperature and basified with 10% aqueous potassium carbonate. Methylene chloride was added to the aqueous mixture and a solid precipitated. The solid was filtered and dried to yield 2.98 g of E-[6-(3-amino-propenyl)-quinazolin-4-yl]-[3-chloro-4-(6-methyl-pyridin-3-yloxy)-phenyl]-amine. ¹H NMR (d₆ DMSO): δ8.62 (s, 1), 8.53 (m, 1), 8.26 (m, 2), 7.99 (m, 1), 7.89 (m, 1), 7.77 (m, 1), 7.30 (m, 3), 6.67 (m, 2), 3.44 (m, 2), 2.47 (s, 3).

E-N-(3-{4-[3-Chloro-4-(6-methyl-pyridin-3-yloxy)-phenylamino]-quinazolin-6-yl}-allyl)-acetamide. A mixture of 14.4 μL (0.25 mmol) of acetic acid and 40.3 mg (0.33 mmol) of dicyclohexylcarbodiimide in 2 mL of methylene chloride were stirred for 10 minutes and treated with 100.3 mg of E-[6-(3-amino-propenyl)-quinazolin-4-yl]-[3-chloro-4-(6-methyl-pyridin-3-yloxy)-phenyl]-amine. The reaction was allowed to stir at room temperature overnight. The precipitate which formed was filtered and chromatographed on silica gel, eluting with 6-10% methanol/chloroform to afford 106 mg of the title compound; mp 254-256° C.; ¹H NMR (d₆ DMSO): δ9.88 (s, 1), 8.58 (s, 1), 8.48 (m, 1), 8.20 (m, 3), 7.95 (m, 1), 7.83 (m, 1), 7.71(d, J=8.7 Hz, 1), 7.24 (m, 2), 7.19 (d, J=8.7 Hz, 1), 6.61 (d, J=16.2 Hz, 1), 6.48 (m, 1), 3.90 (m, 2).

Method H: E—2S-Methoxymethyl-pyrrolidine-1-carboxylic acid (3-{4-[3-methyl-4-(6-methyl-pyridin-3-yloxy)-phenylamino]-quinazolin-6-yl{-allyl)-amide (8):

To a stirred solution of 0.125 (0.31 mmol) of E-[6-(3-amino-propenyl)-quinazolin-4-yl]-[3-methyl-4-(6-methyl-pyridin-3-yloxy)-phenyl]-amine (prepared according to method G) in 1 mL of dichloromethane at 0° C. was added 60.3 μL (0.34 mmol) of Hunig's base followed by dropwise addition of a solution of 48.2 μL (0.34 mmol) of 4-chlorophenyl chloroformate in 1 mL of dichloromethane. The reaction was stirred 30 minutes and evaporated under reduced pressure. The residue was dissolved in 2 mL of dimethyl sulfoxide and 123 μL (0.94 mmol) of (S)-(+)-2-(methoxymethyl)-pyrrolidine was added neat. The reaction was stirred for 3 hours at room temperature. The reaction was quenched into 10% potassium carbonate and extracted with ethyl acetate. The organic layer was washed several times with water and twice with brine. The organic layer was dried over sodium sulfate and reduced to yield the crude material. This material was purified over 90 g of silica gel using 96:4:0.1 chloroform:methanol:ammonium hydroxide as eluent to yield 75 mg (0.14 mmol) of the title compound. ¹HNMR (d₆ DMSO): δ9.83 (s, 1), 8.56 (s, 2), 8.21 (d, 1), 7.95 (d, 1), 7.80 (d, 1), 7.50 (d, 1), 7.25 (m, 2), 7.01 (d, 1), 6.63 (d, 1), 6.53 (m, 1), 3.95 (m, 2), 3.40 (dd, 1), 3.28 (s, 3), 2.49 (s, 3), 2.24 (s, 3), 1.85 (m, 4).

Method I: E-2-Hydroxy-N-(3-{4-[3-methyl-4-(6-methyl-pyridin-3-yloxy)-phenylamino]-quinazolin-6-yl}-allyl)-isobutyramide (9):

To a solution of 0.170 g (0.42 mmol) of E-[6-(3-amino-propenyl)-quinazolin-4-yl]-[3-methyl-4-(6-methyl-pyridin-3-yloxy)-phenyl]-amine (prepared according to method G) in 1 mL of dichloromethane at 0° C. was added 65 μL (0.47 mmol) of triethylamine followed by a solution of 65 μL (0.45 mmol) of 2-acetoxyisobutyryl chloridein 1 mL of dichloromethane. The reaction was stirred at 0° C. for 1 hour. The mixture was quenched with a dropwise addition of 10% potassium carbonate. The aqueous layer was extracted with dichloromethane and the combined organics were washed with brine, dried over sodium sulfate and evaporated. The crude material was purified on 90 g of silica gel eluting with 96:4:0.1 chloroform/methanol/ammonium hydroxide to afford 2-acetoxy-N-(3-{4-[3-methyl-4-(6-methyl-pyridin-3-yloxy)-phenylamino]-quinazolin-6-yl}-allyl)-isobutyramide. A solution of this material in 2 mL of methanol was treated dropwise with a solution of 41 mg (3.02 mmol) of potassium carbonate in 0.5 mL of water. The solution was stirred at room temperature for 1 hour. The reaction was evaporated and the residue was partitioned between water and chloroform. The aqueous layer was extracted twice with chloroform and the combined organics were washed with brine, dried over sodium sulfate and evaporated to yield 100 mg of the title compound (47%). ¹HNMR (d₆ DMSO): δ9.78 (s, 1), 8.50 (s, 1), 8.48 (s, 1), 8.15 (d, 1), 7.95 (m, 3), 7.21 (m, 2), 6.96 ( dt, 1), 3.92 (t, 2), 2.46 (s, 3), 2.1.

The following examples were prepared using the methods described above.

TABLE I

| Example | | | | HPLC |
|---|---|---|---|---|
| No. | Name | Method | LRMS | RT |
| 1 | N-{3-[4-(5-Methyl-6-phenoxy-pyridin-3- | B | 452.2 | 7.10 |
| | ylamino)-quinazolin-6-yl]-prop-2-ynyl}-2- | | | |
| | oxo-propionamide | | | |
| 2 | E-Cyclopropanecarboxylic acid (3-{4-[3- | G | 452.2 | 5.48 |
| | methyl-4-(pyridin-3-yloxy)-phenylamino]- | | | |
| | quinazolin-6-yl}-allyl)-amide | | | |
| 3 | 2-Methoxy-N-(3-{4-[4-(3-methoxy- | B | 483.2 | 6.72 |
| | phenoxy)-3-methyl-phenylamino]- | | | |
| | quinazolin-6-yl}-prop-2-ynyl)-acetamide | G | 485.7 | 5.77 |
| 4 | E-Cyclopropanecarboxylic acid (3-{4-[3- | | | |
| | chloro-4-(6-methyl-pyridin-3-yloxy)- | | | |
| | phenylamino]-quinazolin-6-yl}-allyl)-amide | | | |
| 5 | E-N-(3-{4-[3-Chloro-4-(6-methyl-pyridin-3- | G | 460.0 | 5.01 |
| | yloxy)-phenylamino]-quinazolin-6-yl}-allyl)- | | | |
| | acetamide | | | |
| 6 | E-5-Methyl-isoxazole-3-carboxylic acid (3- | G | 507.2 | 6.04 |
| | {4-[3-methyl-4-(6-methyl-pyridin-3-yloxy)- | | | |
| | phenylamino]-quinazolin-6-yl}-allyl)-amide | | | |
| 7 | E-(3-{4-[3-Methyl-4-(pyridin-3-yloxy)- | G | 442.3 | 5.60 |
| | phenylamino]-quinazolin-6-yl}-allyl)- | | | |
| | carbamic acid methyl ester | | | |
| 8 | 3-Methoxy-pyrrolidine-1-carboxylic acid | | | |
| | (1,1-dimethyl-3-{4-[3-methyl-4-(6-methyl- | | | |
| | pyridin-3-yloxy)-phenylamino]-quinazolin-6- | D | 551.3 | 6.27 |
| | yl}-prop-2-ynyl)-amide | | | |
| 9 | E-2-Methoxy-N-(3-{4-[3-methyl-4-(6- | G | 470.1 | 5.05 |
| | methyl-pyridin-3-yloxy)-phenylamino]- | | | |
| | quinazolin-6-yl}-allyl)-acetamide | | | |
| 10 | 1-Ethyl-3-(3-{4-[3-methyl-4-(pyridin-3- | D | 453.1 | 5.16 |
| | yloxy)-phenylamino]-quinazolin-6-yl}-prop- | | | |
| | 2-ynyl)-urea | | | |
| 11 | E-Cyclopropanecarboxylic acid (3-{4-[3- | G | 466.1 | 5.41 |
| | methyl-4-(6-methyl-pyridin-3-yloxy)- | | | |
| | phenylamino]-quinazolin-6-yl}-allyl)-amide | | | |
| 12 | 1-(3-{4-[3-Chloro-4-(pyridin-3-yloxy)- | | | |
| | phenylamino]-quinazolin-6-yl}-prop-2-ynyl)- | D | 473.2 | 5.45 |
| | 3-ethyl-urea | | | |
| 13 | 2-Dimethylamino-N-(3-{4-[3-methyl-4- | C | 467.3 | 4.15 |
| | (pyridin-3-yloxy)-phenylamino]-quinazolin- | | | |
| | 6-yl}-prop-2-ynyl)-acetamide | | | |
| 14 | [3-Methyl-4-(pyridin-3-yloxy)-phenyl]-(6- | A | 236.6 | 4.35 |
| | piperidin-4-ylethynyl-quinazolin-4-yl)-amine | | | |
| 15 | (3-{4-[3-Methyl-4-(pyridin-3-yloxy)- | B | 440.3 | 5.61 |
| | phenylamino]-quinazolin-6-yl}-prop-2-ynyl)- | | | |
| | carbamic acid methyl ester | | | |
| 16 | 3-Methyl-isoxazole-5-carboxylic acid (3-{4- | B | 505.4 | 6.05 |
| | [3-methyl-4-(6-methyl-pyridin-3-yloxy)- | | | |
| | phenylamino]-quinazolin-6-yl}-prop-2-ynyl)- | | | |
| | amide | | | |

### EXAMPLE 17

The IC₅₀ values for the inhibition of erbB1 receptor autophosphorylation and erbB2 receptor autophophorylation were determined using the in vitro cell assays described above. The following table shows selectivity of the small molecules for the erbB2 tyrosine kinase versus the erbB1 tyrosine kinase in the form of a ratio of erbB2:erbB1 selectivity ratio. The last column shows the potency (IC₅₀) of the each of the small molecules for the erbB2 receptor with the following key: ***<20 nM; **21-50 nM; and * is 51-100 nM. The small molecule compounds shown below are potent and highly selective inhibitors for the erbB2 receptor tyrosine kinase.

| | erbB2/ | | | |
|---|---|---|---|---|
| | erbB1 | | Method of | Example |
| Compound Name | ratio | Potency | prep | # |
| N-{3-[4-(5-Methyl-6-phenoxy-pyridin- | 101 | *** | B | 1 |
| 3-ylamino)-quinazolin-6-yl]-prop-2- | | | | |
| ynyl}-2-oxo-propionamide | | | | |
| E-Cyclopropanecarboxylic acid (3-{4- | | | | |
| [3-methyl-4-(pyridin-3-yloxy)- | 658 | ** | G | 2 |
| phenylamino]-quinazolin-6-yl}-allyl)- | | | | |
| amide | | | | |
| 2-Methoxy-N-(3-{4-[4-(3-methoxy- | 103 | ** | B | 3 |
| phenoxy)-3-methyl-phenylamino]- | | | | |
| quinazolin-6-yl}-prop-2-ynyl)- | | | | |
| acetamide | | | | |
| E-Cyclopropanecarboxylic acid (3-{4- | 142 | ** | G | 4 |
| [3-chloro-4-(6-methyl-pyridin-3- | | | | |
| yloxy)-phenylamino]-quinazolin-6-yl}- | | | | |
| allyl)-amide | | | | |
| E-N-(3-{4-[3-Chloro-4-(6-methyl- | 108 | ** | G | 5 |
| pyridin-3-yloxy)-phenylamino]- | | | | |
| quinazolin-6-yl}-allyl)-acetamide | | | | |
| E-5-Methyl-isoxazole-3-carboxylic | 437 | *** | G | 6 |
| acid (3-{4-[3-methyl-4-(6-methyl- | | | | |
| pyridin-3-yloxy)-phenylamino]- | | | | |
| quinazolin-6-yl}-allyl)-amide | | | | |
| E-(3-{4-[3-Methyl-4-(pyridin-3-yloxy)- | 1133 | ** | G | 7 |
| phenylamino]-quinazolin-6-yl}-allyl)- | | | | |
| carbamic acid methyl ester | | | | |
| 3-Methoxy-pyrrolidin-1-carboxylic | 308 | * | D | 8 |
| acid (1,1-dimethyl-3-{4-[3-methyl-4- | | | | |
| (6-methyl-pyridin-3-yloxy)- | | | | |
| phenylamino]-quinazolin-6-yl}-prop-2- | | | | |
| ynyl)-amide | | | | |
| E-2-Methoxy-N-(3-{4-[3-methyl-4-(6- | 116 | ** | G | 9 |
| methyl-pyridin-3-yloxy)- | | | | |
| phenylamino]-quinazolin-6-yl}-allyl)- | | | | |
| acetamide | | | | |
| 1-Ethyl-3-(3-{4-[3-methyl-4-(pyridin- | 112 | ** | D | 10 |
| 3-yloxy)-phenylamino]-quinazolin-6- | | | | |
| yl}-prop-2-ynyl)-urea | | | | |
| E-Cyclopropanecarboxylic acid (3-{4- | 122 | ** | G | 11 |
| [3-methyl-4-(6-methyl-pyridin-3- | | | | |
| yloxy)-phenylamino]-quinazolin-6-yl}- | | | | |
| allyl)-amide | | | | |
| 1-(3-{4-[3-Chloro-4-(pyridin-3-yloxy)- | 121 | ** | D | 12 |
| phenylamino]-quinazolin-6-yl}-prop-2- | | | | |
| ynyl)-3-ethyl-urea | | | | |
| 2-Dimethylamino-N-(3-{4-[3-methyl- | 182 | *** | C | 13 |
| 4-(pyridin-3-yloxy)-phenylamino]- | | | | |
| quinazolin-6-yl}-prop-2-ynyl)- | | | | |
| acetamide | | | | |
| [3-Methyl-4-(pyridin-3-yloxy)-phenyl]- | 196 | ** | A | 14 |
| (6-piperidin-4-ylethynyl-quinazolin-4- | | | | |
| yl)-amine | | | | |
| (3-{4-[3-Methyl-4-(pyridin-3-yloxy)- | 140 | * | B | 15 |
| phenylamino]-quinazolin-6-yl}-prop-2- | | | | |
| ynyl)-carbamic acid methyl ester | | | | |
| 3-Methyl-isoxazole-5-carboxylic acid | 216 | ** | B | 16 |
| (3-{4-[3-methyl-4-(6-methyl-pyridin-3- | | | | |
| yloxy)-phenylamino]-quinazolin-6-yl}- | | | | |
| prop-2-ynyl)-amide | | | | |

1. A small molecule erbB2 inhibitor, wherein said erbB2 inhibitor has a range of selectivities for erbB2 over erbB1 between 50-1500.

2. The small molecule erbB2 inhibitor of claim 1, wherein said erbB2 inhibitor has a range of selectivities for erbB2 over erbB1 between 60-1200.

3. The small molecule erbB2 inhibitor of claim 2, wherein said erbB2 inhibitor has a range of selectivities for erbB2 over erbB1 between 80-1000.

4. The small molecule erbB2 inhibitor of claim 3, wherein said erbB2 inhibitor has a range of selectivities for erbB2 over erbB1 between 90-500.

5. The small molecule erbB2 inhibitor of claim 4, wherein said erbB2 inhibitor has a range of selectivities for erbB2 over erbB1 between 100-300.

6. The small molecule erbB2 inhibitor of claim 5, wherein said erbB2 inhibitor has a range of selectivities for erbB2 over erbB1 between 110-200.

7. The small molecule erbB2 inhibitor of claim 1, wherein said erbB2 inhibitor has an IC₅₀ of less than about 100 nM.

8. The small molecule erbB2 inhibitor of claim 7, wherein said erbB2 inhibitor has an IC₅₀ of less than about 50 nM.

9. The small molecule erbB2 inhibitor of claim 2, wherein said erbB2 inhibitor has an IC₅₀ of less than about 100 nM.

10. The small molecule erbB2 inhibitor of claim 9, wherein said erbB2 inhibitor has an IC₅₀ of less than about 50 nM.

11. The small molecule erbB2 inhibitor of claim 3, wherein said erbB2 inhibitor has an IC₅₀ of less than about 100 nM.

12. The small molecule erbB2 inhibitor of claim 11, wherein said erbB2 inhibitor has an IC₅₀ of less than about 50 nM.

13. The small molecule erbB2 inhibitor of claim 4, wherein said erbB2 inhibitor has an IC₅₀ of less than about 100 nM.

14. The small molecule erbB2 inhibitor of claim 13, wherein said erbB2 inhibitor has an IC₅₀ of less than about 50 nM.

15. The small molecule erbB2 inhibitor of claim 5, wherein said erbB2 inhibitor has an IC₅₀ of less than about 50 nM.

16. The small molecule erbB2 inhibitor of claim 15, wherein said erbB2 inhibitor has an IC₅₀ of less than about 100 nM.

17. A small molecule erbB2 inhibitor, wherein said erbB2 inhibitor has a range of selectivities for erbB2 over erbB1 between 50-1500 and inhibits growth of tumor cells which overexpress erbB2 receptor in a patient treated with a therapeutically effective amount of said erbB2 inhibitor.

18. The small molecule erbB2 inhibitor of claim 17, wherein said erbB2 inhibitor has a range of selectivities for erbB2 over erbB1 between 60-1200 and inhibits growth of tumor cells which overexpress erbB2 receptor in a patient treated with a therapeutically effective amount of said erbB2 inhibitor.

19. The small molecule erbB2 inhibitor of claim 18, wherein said erbB2 inhibitor has a range of selectivities for erbB2 over erbB1 between 80-1000 and inhibits growth of tumor cells which overexpress erbB2 receptor in a patient treated with a therapeutically effective amount of said erbB2 inhibitor.

20. The small molecule erbB2 inhibitor of claim 19, wherein said erbB2 inhibitor has a range of selectivities for erbB2 over erbB1 between 90-500 and inhibits growth of tumor cells which overexpress erbB2 receptor in a patient treated with a therapeutically effective amount of said erbB2 inhibitor.

21. The small molecule erbB2 inhibitor of claim 20, wherein said erbB2 inhibitor has a range of selectivities for erbB2 over erbB1 between 100-300 and inhibits growth of tumor cells which overexpresses erbB2 receptor in a patient treated with a therapeutically effective amount of said erbB2 inhibitor.

22. The small molecule erbB2 inhibitor of claim 21, wherein said erbB2 inhibitor has a range of selectivities for erbB2 over erbB1 between 110-200 and inhibits growth of tumor cells which overexpresses erbB2 receptor in a patient treated with a therapeutically effective amount of said erbB2 inhibitor.

23. The small molecule erbB2 inhibitor of claim 17, wherein said erbB2 inhibitor has an IC₅₀ of less than about 100 nM.

24. The small molecule erbB2 inhibitor of claim 23, wherein said erbB2 inhibitor has an IC₅₀ of less than about 50 nM.

25. The small molecule erbB2 inhibitor of claim 18, wherein said erbB2 inhibitor has an IC₅₀ of less than about 100 nM.

26. The small molecule erbB2 inhibitor of claim 25, wherein said erbB2 inhibitor has an IC₅₀ of less than about 50 nM.

27. The small molecule erbB2 inhibitor of claim 19, wherein said erbB2 inhibitor has an IC₅₀ of less than about 100 nM.

28. The small molecule erbB2 inhibitor of claim 27, wherein said erbB2 inhibitor has an IC₅₀ of less than about 50 nM.

29. The small molecule erbB2 inhibitor of claim 20, wherein said erbB2 inhibitor has an IC₅₀ of less than about 100 nM.

30. The small molecule erbB2 inhibitor of claim 29, wherein said erbB2 inhibitor has an IC₅₀ of less than about 50 nM.

31. A method of treating abnormal cell growth in a mammal comprising administering to said mammal an amount of a small molecule erbB2 inhibitor that is effective in treating abnormal cell growth and said erbB2 inhibitor has a range of selectivities for erbB2 over erbB1 between 50-1500.

32. The method of claim 31, wherein said erbB2 inhibitor has a range of selectivities for erbB2 over erbB1 between 60-1200.

33. The method of claim 32, wherein said erbB2 inhibitor has a range of selectivities for erbB2 over erbB1 between 80-1000.

34. The method of claim 33, wherein said erbB2 inhibitor has a range of selectivities for erbB2 over erbB1 between 90-500.

35. The method of claim 34, wherein said erbB2 inhibitor has a range of selectivities for erbB2 over erbB1 between 100-300.

36. The method of claim 35, wherein said erbB2 inhibitor has a range of selectivities for erbB2 over erbB1 between 110-200.

37. The method of claim 31, wherein said erbB2 inhibitor has an IC₅₀ of less than about 100 nM.

38. The method of claim 37, wherein said erbB2 inhibitor has an IC₅₀ of less than about 50 nM.

39. The method of claim 32, wherein said erbB2 inhibitor has an IC₅₀ of less than about 100 nM.

40. The method of claim 39, wherein said erbB2 inhibitor has an IC₅₀ of less than about 50 nM.

41. The method of claim 33, wherein said erbB2 inhibitor has an erbB2 inhibitor has an IC₅₀ of less than about 100 nM.

42. The method of claim 41, wherein said erbB2 inhibitor has an IC₅₀ of less than about 50 nM.

43. The method of claim 34, wherein said erbB2 inhibitor has an erbB2 inhibitor has an IC₅₀ of less than about 100 nM.

44. The method of claim 46, wherein said erbB2 inhibitor has an IC₅₀ of less than about 50 nM.

45. The method of claim 38, wherein said erbB2 inhibitor has an IC₅₀ of less than about 100 nM.

46. The method of claim 48, wherein said erbB2 inhibitor has an IC₅₀ of less than about 50 nM.

47. The method of claim 39, wherein said erbB2 inhibitor has an IC₅₀ of less than about 100 nM.

48. The method of claim 50, wherein said erbB2 inhibitor has an IC₅₀ of less than about 50 nM.

49. The method of claim 31, wherein said erbB2 inhibitor is selected from the group consisting of:
N-{3-[4-(5-Methyl-6-phenoxy-pyridin-3-ylamino)-quinazolin-6-yl]-prop-2-ynyl}-2-oxo-propionamide
E-cyclopropanecarboxylic acid (3-{4-[3-methyl-4-(pyridin-3-yloxy)-phenylamino]-quinazolin-6-yl}-allyl)-amide
2-methoxy-N-(3-{4-[4-(3-methoxy-phenoxy)-3-methyl-phenylamino]-quinazolin-6-yl}-prop-2-ynyl)-acetamide
E-cyclopropanecarboxylic acid (3-{4-[3-chloro-4-(6-methyl-pyridin-3-yloxy)-phenylamino]-quinazolin-6-yl}-allyl)-amide
E-N-(3-{4-[3-chloro-4-(6-methyl-pyridin-3-yloxy)-phenylamino]-quinazolin-6-yl}-allyl )-acetamide
E-5-methyl-isoxazole-3-carboxylic acid (3-{4-[3-methyl-4-(6-methyl-pyridin-3-yloxy)-phenylamino]-quinazolin-6-yl}-allyl)-amide
E-3-{4-[3-methyl-4-(pyridin-3-yloxy)-phenylamino]-quinazolin-6-yl}-allyl)-carbamic acid methyl ester
3-methoxy-pyrrolidine-1-carboxylic acid (1,1-dimethyl-3-{4-[3-methyl-4-(6-methyl-pyridin-3-yloxy)-phenylamino]-quinazolin-6-yl}-prop-2-ynyl)-amide
E-2-methoxy-N-(3-{4-[3-methyl-4-(6-methyl-pyridin-3-yloxy)-phenylamino]-quinazolin-6-yl}-allyl)-acetamide
1-ethyl-3-(3-{4-[3-methyl-4-(pyridin-3-yloxy)-phenylamino]-quinazolin-6-yl}-prop-2-ynyl)-urea
E-cyclopropanecarboxylic acid (3-{4-[3-methyl-4-(6-methyl-pyridin-3-yloxy)-phenylamino]-quinazolin-6-yl}-allyl)-amide
1-(3-{4-[3-chloro-4-(pyridin-3-yloxy)-phenylamino]-quinazolin-6-yl}-prop-2-ynyl)-3ethyl-urea
2-dimethylamino-N-(3-{4-[3-methyl-4-(pyridin-3-yloxy)-phenylamino]-quinazolin-6-yl}-prop-2-ynyl)-acetamide
3-methyl-4-(pyridin-3-yloxy)-phenyl]-(6-piperidin-4-ylethynyl-quinazolin-4-yl)-amine
(3-{4-[3-methyl-4-(pyridin-3-yloxy)-phenylamino]-quinazolin-6-yl}-prop-2-ynyl)-carbamic acid methyl ester
3-methyl-isoxazole-5-carboxylic acid (3-{4-[3-methyl-4-(6-methyl-pyridin-3-yloxy)-phenylamino]-quinazolin-6-yl}-prop-2-ynyl)-amide,
and the pharmaceutically acceptable salts, prodrugs and solvates of the foregoing compounds.

50. The method of claim 49, wherein said erbB2 inhibitor is selected from the group consisting of:
E-cyclopropanecarboxylic acid (3-{4-[3-methyl-4-(pyridin-3-yloxy)-phenylamino]-quinazolin-6-yl}-allyl)-amide
E-5-methyl-isoxazole-3-carboxylic acid (3-{4-[3-methyl-4-(6-methyl-pyridin-3-yloxy)-phenylamino]-quinazolin-6-yl}-allyl)-amide
E-(3-{4-[3-methyl-4-(pyridin-3-yloxy)-phenylamino]-quinazolin-6-yl}-allyl)-carbamic acid methyl ester
3-methoxy-pyrrolidine-1-carboxylic acid (1,1-dimethyl-3-{4-[3-methyl-4-(6-methyl-pyridin-3-yloxy)-phenylamino]-quinazolin-6-yl}-prop-2-ynyl)-amide
3-methyl-isoxazole-5-carboxylic acid (3-{4-[3-methyl-4-(6-methyl-pyridin-3-yloxy)-phenylamino]-quinazolin-6-yl}-prop-2-ynyl)-amide,
and the pharmaceutically acceptable salts, prodrugs and solvates of the foregoing compounds.

51. The method of claim 50, wherein said erbB2 inhibitor is selected from the group consisting of:
E-cyclopropanecarboxylic acid (3-{4-[3-methyl-4-(pyridin-3-yloxy)-phenylamino]-quinazolin-6-yl}-allyl)-amide
E-(3-{4-[3-methyl-4-(pyridin-3-yloxy)-phenylamino]-quinazolin-6-yl)-allyl)-carbamic acid methyl ester
and the pharmaceutically acceptable salts, prodrugs and solvates of the foregoing compounds.

52. A method for the treatment of abnormal cell growth in a mammal comprising administering to said mammal an amount of a compound of claim 1 that is effective in treating abnormal cell growth.

53. A method according to claim 52, wherein said abnormal cell growth is cancer.

54. The method according to claim 53 wherein said cancer is selected from lung cancer, non small cell lung (NSCL), bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), colorectal cancer (CRC), primary CNS lymphoma, spinal axis tumors, brain stem glioma, pituitary adenoma, or a combination of one or more of the foregoing cancers.

55. The method according to claim 54, wherein said cancer is selected from breast cancer, colon cancer, ovarian cancer, non small cell lung (NSCL) cancer, colorectal cancer (CRC), prostate cancer, bladder cancer, renal cancer, gastric cancer, endometrial cancer, head and neck cancer, and esophagel cancer.

56. The method according to claim 55, wherein said cancer is selected from renal cancer, gastric cancer, colon cancer, breast cancer, and ovarian cancer.

57. The method according to claim 56, wherein said cancer is selected from colon cancer, breast cancer or ovarian cancer.

58. The method according to claim 57, wherein said cancer is breast cancer.

59. The method according to claim 57, wherein said cancer is ovarian cancer.

60. The method according to claim 57, wherein said cancer is colon cancer.

61. A method for the treatment of abnormal cell growth in a mammal which comprises administering to said mammal an amount of a compound of claim 1 that is effective in treating abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

62. The method of claim 61, which comprises administering to said mammal an amount of a compound of claim 1 that is effective in treating abnormal cell growth in combination with a cytotoxic.

63. The method of claim 62, which comprises administering to said mammal an amount of a compound of claim 1 that is effective in treating abnormal cell growth in combination with Taxol®.

64. A method for the treatment of abnormal cell growth in a mammal which comprises administering to said mammal an amount of a compound of claim 1 that is effective in treating abnormal cell growth in combination with a compound selected from the group consisting of Cyclophosphamide, 5-Fluorouracil, Floxuridine, Gemcitabine, Vinblastine, Vincristine, Daunorubicin, Doxorubicin, Epirubicin, Tamoxifen, Methylprednisolone, Cisplatin, Carboplatin, CPT-11, gemcitabine, paclitaxel, and docetaxel.

65. The method of claim 64, comprises administering to said mammal an amount of a compound of claim 1 that is effective in treating abnormal cell growth in combination with a compound selected from the group consisting Tamoxifen, Cisplatin, Carboplatin, paclitaxel and docetaxel.

66. A method of treating abnormal cell growth in a mammal comprising administering to said mammal a small molecule erbB2 inhibitor in an amount that is effective in treating abnormal cell growth and said erbB2 inhibitor has a range of selectivities for erbB2 over erbB1 between 50-1500 as measured by an in vitro cell assay.

67. The method of claim 66, wherein said erbB2 inhibitor has a range of selectivities for erbB2 over erbB1 between 60-1200.

68. The method of claim 67, wherein said erbB2 inhibitor has a range of selectivities for erbB2 over erbB1 between 80-1000.

69. The method of claim 68, wherein said erbB2 inhibitor has a range of selectivities for erbB2 over erbB1 between 90-500.

70. The method of claim 69, wherein said erbB2 inhibitor has a range of selectivities for erbB2 over erbB1 between 100-300.

71. The method of claim 70, wherein said erbB2 inhibitor has a range of selectivities for erbB2 over erbB1 between 110-200.

72. The method of claim 66, wherein the abnormal cell growth is cancer.

73. The method of claim 72, wherein the cancer is colon, breast or ovarian cancer.

74. A method for treating a mammal having a disease characterized by an overexpression of erbB2, comprising administering to the mammal a small molecule erbB2 inhibitor in an amount that is effective in treating a disease characterized by the overexpression of erbB2 and said erbB2 inhibitor has a range of selectivities for erbB2 over erbB1 between 50-1500.

75. The method of claim 74, wherein said erbB2 inhibitor has a range of selectivities for erbB2 over erbB1 between 60-1200.

76. The method of claim 75, wherein said erbB2 inhibitor has a range of selectivities for erbB2 over erbB1 between 80-1000.

77. The method of claim 76, wherein said erbB2 inhibitor has a range of selectivities for erbB2 over erbB1 between 90-500.

78. The method of claim 77, wherein said erbB2 inhibitor has a range of selectivities for erbB2 over erbB1 between 100-300.

79. The method of claim 78, wherein said erbB2 inhibitor has a range of selectivities for erbB2 over erbB1 between 110-200.

80. The method of claims 74, 75, 76, 77, 78, and 79, wherein said erbB2 inhibitor has an IC₅₀ of less than about 100 nM.

81. The method of claim 80, wherein said erbB2 inhibitor has an IC₅₀ of less than about 50 nM.

82. A method for treating a mammal having cancer characterized by an overexpression of erbB2, comprising administering to the mammal a small molecule erbB2 inhibitor in an amount that is effective in treating said cancer characterized by the overexpression of erbB2 and said erbB2 inhibitor has a range of selectivities for erbB2 over erbB1 between 50-1500.

83. The method of claim 82, wherein said erbB2 inhibitor has a range of selectivities for erbB2 over erbB1 between 60-1200.

84. The method of claim 83, wherein said erbB2 inhibitor has a range of selectivities for erbB2 over erbB1 between 80-1000.

85. The method of claim 84, wherein said erbB2 inhibitor has a range of selectivities for erbB2 over erbB1 between 90-500.

86. The method of claim 85, wherein said erbB2 inhibitor has a range of selectivities for erbB2 over erbB1 between 100-300.

87. The method of claim 86, wherein said erbB2 inhibitor has a range of selectivities for erbB2 over erbB1 between 110-200.

88. The method of claims 82, 83, 84, 85, 86, and 87 wherein said erbB2 inhibitor has an IC₅₀ of less than about 100 nM.

89. The method of claim 88, wherein said erbB2 inhibitor has an IC₅₀ of less than about 50 nM.

90. A method for inducing cell death comprising exposing a cell which overexpresses ErbB2 to an effective amount of an erbB1-sparing erbB2 inhibitor.

91. The method of claim 90, wherein the cell is a cancer cell.

92. The method of claim 90, wherein the cell is in a mammal.

93. The method of claim 92, wherein the mammal is a human.

94. The method of claim 90, further comprising exposing the cell to a growth inhibitory agent.

95. The method of claim 90, further comprising exposing the cell to a chemotherapeutic agent.

96. The method of claim 90, further comprising exposing the cell to radiation.

97. A method of treating cancer in a human, wherein the cancer expresses the erbB2 receptor, comprising administering to the human a therapeutically effective amount of an erbB2 inhibitor that has reduced affinity for the erbB1 receptor.

98. The method of claim 97, wherein the cancer is not characterized by overexpression of erbB1 receptor.

99. The method of claim 97, wherein the cancer is characterized by overexpression of the erbB1 and erbB2 receptor.
